(19) United States
(12) Patent Application Publication
(10) Pub. No.: US 2003/0139443 A1
(43) Pub. Date: Jul. 24, 2003

(54) Use of tachykinin antagonists, including NK-1 receptor antagonists, to modify unwanted behavior in dogs, cats and horses

(76) Inventors: Brian Scott Bronk, Gales Ferry, CT (US); Mary Anne Hickman, East Lyme, CT (US); Carolyn Rose Kilroy, Old Lyme, CT (US)

Correspondence Address: PFIZER INC, 150 EAST 42ND STREET, 5TH FLOOR - STOP 49, NEW YORK, NY, 10017-5612, US

(21) Appl. No.: 10/199,284

(22) Filed: Jul. 19, 2002

Related U.S. Application Data
(60) Provisional application No. 60/306,692, filed on Jul. 20, 2001.

(51) Int. Cl. A61K 31/454; A61K 31/49; A61K 31/445

(52) U.S. Cl. 514/305; 514/317; 514/326

(57) ABSTRACT

The present invention relates to a method of treating abnormal anxiety behavior in companion animals comprising administering to a companion animal in need thereof a therapeutically effective amount of an NK-1 receptor antagonist.

### BACKGROUND OF THE INVENTION

Anxiety behaviors in companion animals can be devastating conditions that, in the absence of treatment, may result in relinquishment to a humane society or shelter, abandonment, or euthanasia. For example, a study of dogs in animal shelters in the Netherlands showed that 50% of dogs that found new homes were returned to a shelter. See J. van der Borg, W. J. Netto and D. J. Planta, “Behavioural Testing of Dogs in Animal Shelters to Predict Problem Behavior,” Applied Animal Behaviour Science 32 (1991), pp. 237-251. One frequently cited reason for returning a dog was behavior attributed to separation anxiety.

Anxiety behaviors can be common disorders in some species of companion animals. For example, it has been estimated that in the average veterinary practice in the US up to 14% of canine patients exhibit one or more signs of separation anxiety. K. Overall, “Understanding Canine Separation Anxiety.” Elimination, destruction, and vocalization are the most commonly reported behaviors associated with separation anxiety. Id. Other behaviors associated with anxiety include salivation, anorexia, and lethargy. Id.

Excessive barking is a recognized behavior problem in dogs. Soraya Juarbe-Diaz has suggested treating excessive barking by limited use of psychotropic medication after a thorough patient evaluation. S. Juarbe-Diaz, “Assessment and Treatment of Excessive Barking in the Domestic Dog,” Progress in Companion Animal Behavior 27 (May 1997), pp. 515-532. Juarbe-Diaz recommends the following psychoactive drugs as adjuncts to behavior modification in the treatment of selected cases of nuisance barking: amitriptyline, buspirone, clomipramine, and fluoxetine. Id.

It is desirable to provide a pharmaceutical therapy to treat abnormal anxiety behaviors in companion animals and thereby to allow the afflicted animal to remain in its owner's home or as a pet.

To develop a pharmaceutical therapy to treat abnormal anxiety behaviors, it is advantageous to have an accurate, reproducible, and safe method for evaluating whether a test compound hasanxiolytic activity. Angels et al. studied human avoidance as a measure of drug effects on nervous pointer dogs. Angels et al., “Assessment of Pointer Dog Behavior,” Pav. J. Biol. Sci. 17 (April-June 1982), pp. 84-88. Angels et al. describe a “Human Interaction Test” in which positive and negative scores are assigned to different behaviors involving interaction with humans. Van der Borg et al. teach a set of behavioral tests to be administered to dogs in shelters to improve the matching between dog and future owner. Van der Borg et al., at 237.

The tachykinins, substance P, neurokinin A and neurokinin B are structurally similar members of a family of neuropeptides that is believed to be involved in anxiety behaviors in mammals. The involvement of substance P and other tachykinins in the pathophysiology of numerous diseases has been amply demonstrated in the art. For instance, substance P has been shown to be involved in the transmission of pain or migraine (see B. E. B. Sandberg et al., J. Med. Chem. 25 (1982) 1009), as well as in central nervous system disorders such as anxiety and schizophrenia, in respiratory and inflammatory diseases such as asthma and rheumatoid arthritis, respectively, in rheumatic diseases such as fibrositis, and in gastrointestinal disorders and diseases of the GI tract such as ulcerative colitis and Crohn's disease, etc. (see D. Regoli in “Trends in Cluster Headache,” edited by F. Sicuteri et al., Elsevier Scientific Publishers, Amsterdam, pp. 85-95 (1987)).

Substance P is known to bind to the neurokinin 1 (NK-1) receptor. NK-1 receptors have been isolated and characterized.

NK-1 receptor antagonists are being developed for the treatment of disorders associated with an excess or imbalance of tachykinins, and particularly of substance P. For example, WO99/07375 discloses the use of NK-1 receptor antagonists for the treatment or prevention of aggressive behavior. U.S. Pat. No. 6,117,855 and WO 98/15277 disclose combination therapy of an NK-1 receptor antagonist and an anti-depressant or anti-anxiety agent to treat anxiety or depression. Specific tachykinin receptor antagonists are described in WO 96/10568 for the treatment of a multitude of disorders, including anxiety, depression, psychosis and schizophrenia. Similarly, WO 00/35915 discloses piperazine derivatives to treat tachykinin-mediated diseases such as anxiety disorders. Other NK-1 receptor antagonists are identified in U.S. Pat. No. 5,773,450, “Fluoroalkoxybenzylamino derivatives of nitrogen containing heterocycles,” issued Jun. 30, 1998 and U.S. Pat. No. 6,222,038, “Quinuclidine derivatives,” issued Apr. 24, 2001. All patents, articles and other references referred to herein are hereby incorporated herein in their entireties.

Although many NK-1 receptor antagonists have been described and their usefulness to treat tachykinin-related disorders including anxiety has been asserted, disclosure of specific behaviors in companion animals that can be altered by administration of an NK-1 receptor antagonist is not believed to have been provided heretofore.

An object of the invention is to provide a pharmaceutical therapy for companion animals to reduce or prevent unwanted behaviors associated with NK-1 receptor activity. Another object of the present invention is to provide a pharmaceutical therapy for companion animals to treat abnormal anxiety behaviors.

Another object of the present invention is to provide a pharmaceutical therapy for companion animals to treat one or more of the following behaviors: abnormal vocalization (barking, crying, growling, howling and whining); hyperactivity (jumping, pacing, circling, increased inquisitiveness, hypervigilance, and tremors); destruction (chewing, digging, and escape behaviors); abnormal sleep (disrupted sleep, insomnia, and increased sleep); abnormal feeding (anorexia, dysorexia, polyphagia, and obesity); abnormal drinking (polydipsia); abnormal grooming (excessive licking, chewing, and nibbling); abnormal elimination (vomiting, diarrhea, and polyuria); abnormal fears and phobias (loud noises, fireworks, and thunder); and socialization disorders (fear of strangers, dogs and selected objects).

Another object of the present invention is to provide a method of screening a test compound to determine anxiolytic activity in dogs.

### SUMMARY OF THE INVENTION

The present invention relates to a method of treating abnormal anxiety behavior in companion animals comprising administering to a companion animal in need thereof a therapeutically effective amount of an NK-1 receptor antagonist.

The present invention relates to a method of treating abnormal anxiety behavior in a companion animal in which the method comprises evaluating the companion animal for the exhibition of an abnormal anxiety behavior, determining that the companion animal exhibits the abnormal anxiety behavior and thus is in need of treatment, and administering to the companion animal a therapeutically effective amount of an NK-1 receptor antagonist for a time sufficient to reduce or eliminate the abnormal anxiety behavior.

The present invention also provides for the use of an NK-1 receptor antagonist in the manufacture of a medicament for the treatment of abnormal anxiety behavior in companion animals.

The present invention also provides a method of screening a test compound to determine anxiolytic activity in dogs comprising (a) selecting a dog exhibiting anxiety behavior; (b) administering the test compound to the dog; (c) separating the dog from views of other dogs and of humans; (d) measuring a first duration of time, the first duration of time being the time within a separation period of a fixed duration during which the anxiety behavior is exhibited; and (e) comparing the first duration of time with a second duration of time, wherein the second duration of time is the time within a separation period of the fixed duration that the anxiety behavior is exhibited in the dog when the dog has not received the test compound for at least forty-eight hours. If the first duration of time is less than the second duration of time, the test compound is determined to have anxiolytic activity.

### DETAILED DESCRIPTION OF THE INVENTION

The present invention relates to a method of treating abnormal anxiety behavior such as vocalization, hyperactivity, destruction, abnormal sleep, abnormal feeding, abnormal drinking, abnormal grooming, abnormal elimination, abnormal fears and phobias, and socialization disorders in companion animals comprising administering to a companion animal in need thereof a therapeutically effective amount of an NK-1 receptor antagonist.

Preferably, the invention is directed to a method of treating vocalization, hyperactivity, destruction, abnormal feeding, and abnormal elimination in companion animals comprising administering to a companion animal in need thereof a therapeutically effective amount of an NK-1 receptor antagonist.

More preferably, the invention is directed to a method of treating vocalization, hyperactivity, and destruction in companion animals comprising administering to a companion animal in need thereof a therapeutically effective amount of an NK-1 receptor antagonist.

The methods of treatment of the present invention are administered to animals in need thereof. Such animals have exhibited one or more abnormal anxiety behaviors, have been evaluated or diagnosed as exhibiting this behavior, are in need of treatment to reduce or eliminate this behavior, and are treated at a dosage and for a period of time sufficient to reduce or eliminate this behavior.

As used herein, the term “treat” means to reduce or eliminate undesirable behaviors in a patient in need thereof.

As used herein, the term “companion animal” includes dogs, cats, and horses, and preferably is a dog.

The NK-1 receptor antagonist is preferably selected from the group consisting of:

(2S,3S)-3-(5-tert-butyl-2-methoxybenzyl)amino-2-(3-trifluoromethoxyphenyl)piperidine;

(2S,3S)-3-(2-isopropoxy-5-trifluoromethoxybenzyl)amino-2-phenyl-piperidine;

(2S,3S)-3-(2-ethoxy-5-trifluoromethoxybenzyl)amino-2-phenyl-piperidine;

(2S,3S)-3-(2-methoxy-5-trifluoromethoxybenzyl)-amino-2-phenylpiperidine;

(2S,3S)-3(-5-tert-butyl-2-trifluoromethoxybenzyl)amino-2-phenylpiperidine;

2-(diphenylmethyl)-N-(2-methoxy-5-trifluoromethoxy-phenyl)methyl-1-azabicyclo[2.2.2]octan-3-amine;

(2S,3S)-3-[5-chloro-2-(2,2,2-trifluoroethoxy)-benzyl]amino-2-phenylpiperidine;

(2S,3S)-3-(5-tert-butyl-2-trifluoromethoxybenzyl)amino-2-phenylpiperidine;

(2S,3S)-3-(2-isopropoxy-5-trifluoromethoxybenzyl)amino-2-phenylpiperidine;

(2S,3S)-3-(2-difluoromethoxy-5-trifluoromethoxybenzyl)-amino-2-phenylpiperidine;

(2S,3S)-2-phenyl-3-[2-(2,2,2-trifluoroethoxybenzyl)-aminopiperidine;

(2S,3S)-2-phenyl-3-(2-trifluoromethoxybenzyl)]aminopi-peridine;

cis-3-(2-chlorobenzylamino)-2-phenylpiperidine;

cis-3-(2-trifluoromethylbenzylamino)-2-phenyl-piperidine;

cis-3-(2-methoxybenzylamino)-2-(2-fluorophenyl)-piperidine;

cis-3-(2-methoxybenzylamino)-2-(2-chlorophenyl)-piperidine;

cis-3-(2-methoxybenzylamino)-2-(2-methylphenyl)-piperidine;

cis-3-(2-methoxybenzylamino)-2-(3-methoxyphenyl)-piperidine;

cis-3-(2-methoxybenzylamino)-2-(3-fluorophenyl)-piperidine;

cis-3-(2-methoxybenzylamino)-2-(3-chlorophenyl)-piperidine;

cis-3-(2-methoxybenzylamino)-2-phenylpiperidine;

cis-3-(2-methoxybenzylamino)-2-(3-methylphenyl)-piperidine;

cis-3-(2-methoxybenzylamino)-2-(4-fluorophenyl)-piperidine;

cis-3-(2-methoxybenzylamino)-2-(3-thienyl)-piperidine;

cis-3-(2-methoxybenzylamino)-2-phenylazacyclo-heptane;

3-(2-methoxybenzylamino)-4-methyl-2-phenylpiperidine;

3-(2-methoxybenzylamino)-5-methyl-2-phenylpiperidine;

3-(2-methoxybenzylamino)-6-methyl-2-phenylpiperidine;

(2S,3S)-3-(2-methoxybenzylamino)-2-phenylpiperidine;

(2S,3S)-1-(5-carboethoxypent-1-yl)-3-(2-methoxybenzyl-amino)-2-phenylpiperidine;

(2S,3S)-1-(6-hydroxy-hex-1-yl)-3-(2-methoxybenzyl-amino)-2-phenylpiperidine;

(2S,3S)-1-(4-hydroxy-4-phenylbut-1-yl)-3-(2-methoxy-benzylamino)-2-phenylpiperidine;

(2S,3S)-1-(4-oxo-4-phenylbut-1-yl)-3-(2-methoxybenzyl-amino)-2-phenylpiperidine;

(2S,3S)-1-(5,6-dihydroxyhex-1-yl)-3-(2-methoxybenzyl-amino)-2-phenylpiperidine;

cis-3-(5-fluoro-2-methoxybenzylamino)-2-phenyl-piperidine;

(2S,3S)-1-[4-(4-fluorophenyl)-4-oxobut-1-yl]-3-(2-methoxybenzylamino)-2-phenylpiperidine;

(2S,3S)-1-[4-[4-fluorophenyl)-4-hydroxybut-1-yl]-3-(2-methoxybenzylamino)-2-phenylpiperidine;

cis-3-(2-methoxy-5-methylbenzylamino)-2-phenyl-piperidine;

(2S,3S)-1-(4-benzamidobut-1-yl)-3-(2-methoxybenzyl-amino)-2-phenylpiperidine;

cis-3-(2-methoxynaphth-1-ylmethylamino)-2-phenyl-piperidine;

(2S,3S)-3-(2-methoxybenzylamino)-1-(5-N-methyl-carboxamidopent-1-yl)-2-phenylpiperidine;

(2S,3S)-1-(4-cyanobut-1-yl)-3-(2-methoxybenzylamino)-2-phenylpiperidine;

(2S,3S)-1-[4-(2-naphthamido)but-1-yl]-3-(2-methoxy-benzylamino)-2-phenylpiperidine;

(2S,3S)-1-(5-benzamidopent-1-yl)-3-(2-methoxybenzyl-amino)-2-phenylpiperidine;

(2S,3S)-1-(5-aminopent-1-yl)-3-(2-methoxybenzylamino)-2-phenylpiperidine;

(2S,3S)-3-(5-chloro-2-methoxybenzylamino)-2-phenyl-piperidine;

(2S,3S)-3-(2,5-dimethoxybenzylamino)-2-phenyl-piperidine;

cis-3-(3,5-difluoro-2-methoxybenzylamino)-2-phenyl-piperidine;

cis-3-(4,5-difluoro-2-methoxybenzylamino)-2-phenyl-piperidine;

cis-3-(2,5-dimethoxybenzylamino)-1-[4-(4-fluorophenyl)-4-oxobut-1-yl]-2-phenylpiperidine;

cis-3-(5-chloro-2-methoxybenzylamino)-1-(5,6-dihydroxyhex-1-yl)-2-phenylpiperidine;

cis-1-(5,6-dihydroxyhex-1-yl)-3-(2,5-dimethoxy-benzylamino)-2-phenylpiperidine;

cis-2-phenyl-3-[-2(prop-2-yloxy)benzylamino]piperidine;

cis-3-(2,5-dimethoxybenzyl)amino-2-(3-methoxy-phenyl)piperidine hydro-chloride;

cis-3-(5-chloro-2-methoxybenzyl)amino-2-(3-methoxy-phenyl)piperidine dihydrochloride;

cis-3-(5-chloro-2-methoxybenzyl)amino-2-(3-chloro-phenyl)piperidine dihydrochloride;3-(2-methoxybenzylamino)-2,4-diphenylpiperidine;

cis-3-(2-methoxybenzylamino)-2-phenylpyrrolidine;

(2S,3S)-3-(5-ethyl-2-methoxybenzyl)amino-2-phenyl-piperidine;

(2S,3S)-3-(5-n-butyl-2-methoxybenzyl)amino-2-phenyl-piperidine;

(2S,3S)-3-(2-methoxy-5-n-propylbenzyl)amino-2-phenyl-piperidine;

(2S,3S)-3-(5-isopropyl-2-methoxybenzyl)amino-2-phenyl-piperidine;

(2S,3S)-3-(5-s-butyl-2-methoxybenzyl)amino-2-phenyl-piperidine;

(2S,3S)-3-(5-t-butyl-2-methoxybenzyl)amino-2-phenyl-piperidine;

(2S,3S)-3-(2-methoxy-5-phenylbenzyl)amino-2-phenyl-piperidine;

2,4-dimethylthiazole-5-sulfonic acid[4-methoxy-3-((2S,3S)-2-phenylpiperidin-3-ylaminomethyl)phenyl]-methylamide;

N-(4,5-dimethylthiazol-2-yl)-N-[4-methoxy-3-((2S,3S)-2-phenylpiperidin-3-yl-aminomethyl)phenyl]-methanesulfonamide;

{5-[(4,5-dimethylthiazol-2-yl)methylamino]-2-methoxybenzyl}-((2S,3S)-2-phenylpiperidin-3-yl)amine;

{5-(4,5-dimethylthiazol-2-ylamino)-2-methoxybenzyl}-((2S,3S)-2-phenylpiperidin-3-ylamine;

4,5-dimethylthiazole-2-sulfonic acid methyl-[3-((2S,3S)-2-phenylpiperidin-3-ylamino-methyl)-4-trifluoromethoxyphenyl]-amide;

2,4-dimethylthiazole-5-sulfonic acid [4-isopropoxy-3-((2S,3S)-2-phenylpiperidin-3-ylaminomethyl)phenyl]-methylamide;

2,4-dimethylthiazole-5-sulfonic acid [4-isopropoxy-3-((2S,3S)-2-phenylpiperidin-3-yl-aminomethyl)phenyl]-isopropylamide;

2,4-dimethylthiazole-5-sulfonic acid [4-methoxy-3-((2S,3S)-2-phenylpiperidin-3-ylamino-methyl)phenyl]-isopropylamide;

2,4-dimethylthiazole-5-sulfonic acid [4-methoxy-3-((2S,3S)-2-phenylpiperidin-3-ylaminomethyl)phenyl]-isobutylamide;

2,4-dimethylthiazole-5-sulfonic acid [4-isopropoxy-3-((2S,3S)-2-phenylpiperidin-3-ylaminomethyl)phenyl]-isobutylamide;

(2S,3S)-N-(5-isopropyl-2-methoxyphenyl)methyl-2-diphenylmethyl-1-azabicyclo[2.2.2]octan-3-amine;

(2S,3S)-N-(5-tert-butyl-2-methoxyphenyl)methyl-2-diphenylmethyl-1-azabicyclo[2.2.2]octan-3-amine;

(2S,3S)-N-(5-methyl-2-methoxyphenyl)methyl-2-diphenylmethyl-1-azabicyclo[2.2.2]octan-3-amine;

(2S,3S)-N-(5-ethyl-2-methoxyphenyl)methyl-2-diphenylmethyl-1-azabicyclo[2.2.2]octan-3-amine;

(2S,3S)-N-(5-isopropyl-2-methoxyphenyl)methyl-2-diphenylmethyl-1-azabicyclo[2.2.2]octan-3-amine;

(2S,3S)-N-(5-sec-butyl-2-methoxyphenyl)methyl-2-diphenylmethyl-1-azabicyclo[2.2.2]octan-3-amine;

(2S,3S)-N-(5-n-propyl-2-methoxyphenyl)methyl-2-diphenylmethyl-1-azabicyclo[2.2.2]octan-3-amine;

(3R,4S,5S,6S)-N,N-diethyl-5-(5-isopropyl-2-methoxy-benzylamino)-6-diphenylmethyl-1-azabicyclo[2.2.2]octane-3-carboxamide;

(3R,4S,5S,6S)-N,N-diethyl-5-(2,5-dimethoxybenzylamino)-6-diphenylmethyl-1-azabicyclo[2.2.2]octane-3-carboxamide;

(3R,4S,5S,6S)-5-(5-isopropyl-2-methoxybenzylamino)-6-diphenylmethyl-1-azabicyclo[2.2.2]octane-3-carboxylic acid;

(3R,4S,5S,6S)-5-(2-methoxy-2-methylthiobenzylamino)-6-diphenylmethyl-1-azabicyclo[2.2.2]octane-3-carboxylic acid;

(3R,4S,5S,6S)-5-(2,5-dimethoxybenzylamino)-6-diphenyl-methyl-1-azabicyclo-[2.2.2]octane-3-carboxylic acid;

(3R,4S,5S,6S)-5-(2-methoxy-5-methylbenzylamino)-6-diphenylmethyl-1-azabicyclo[2.2.2]octane-3-carboxylic acid;

(3R,4S,5S,6S)-5-(5-ethyl-2-methoxybenzylamino)-6-diphenylmethyl-1-azabicyclo[2.2.2]octane-3-carboxylic acid;

(3R,4S,5S,6S)-5-(2-methoxyl-5-n-propylbenzylamino)-6-diphenylmethyl-1-azabicyclo[2.2.2]octane-3-carboxylic acid;

(3R,4S,5S,6S)-5-(5-sec-butyl-2-methoxybenzylamino)-6-diphenylmethyl-1-azabicyclo[2.2.2]octane-3-carboxylic acid;

(3R,4S,5S,6S)-5-(5-N-methyl-methanesulfonylamino-2-methoxy-benzylamino)-6-diphenylmethyl-1-azabicyclo[2.2.2]octane-3-carboxylic acid;

(3R,4S,5S,6S)-5-(2-methoxy-5-methylsulfinylbenzyl-amino)-6-diphenylmethy1-1-azabicyclo[2.2.2]octane-3-carboxylic acid;

(3R,4S,5S,6S)-5-(2-methoxy-5-trifluoromethoxybenzyl-amino)-6-diphenylmethyl-1-azabicyclo[2.2.2]octane-3-carboxylic acid;

(3R,4S,5S,6S)-5-(2-methoxy-5-methylsulfonylbnezyl-amino)-6-diphenylmethy-1-azabicyclo[2.2.2]octane-3-carboxylic acid;

(3R,4S,5S,6S)-5-(5-dimethylamino-2-methoxybenzylamino)-6-diphenylmethyl-1-azabicyclo[2.2.2]octane-3-carboxylic acid;

(3R,4S,5S,6S)-5-(5-isopropyl-2-methoxybenzylamino)-6-diphenylmethyl-1-azabicyclo[2.2.2]octane-2-carboxylic acid;

(3 R,4S,5S,6S)-5-(2-methoxy-5-methylthiobenzylamino)-6-diphenylmethyl-1-azabicyclo[2.2.2]octane-2-carboxylic acid;

(3R,4S,5S,6S)-5-(2,5-dimethoxybenzylamino)-6-diphenylmethyl-1-azabicyclo[2.2.2]octane-2-carboxylic acid;

(3R,4S,5S,6S)-5-(2-methoxy-5-methylbenzylamino)-6-diphenylmethyl-1-azabicyclo[2.2.2]octane-2-carboxylic acid;

(3R,4S,5S,6S)-5-(5-ethyl-2-methoxybenzylamino)-6-diphenylmethyl-1-azabicyclo[2.2.2]octane-2-carboxylic acid;

(3R,4S,5S,6S)-5-(2-methoxyl-5-n-propylbenzylamino)-6-diphenylmethyl-1-azabicyclo[2.2.2]octane-2-carboxylic acid;

(3R,4S,5S,6S)-5-(5-sec-butyl-2-methoxybenzylamino)-6-diphenylmethyl-1-azabicyclo[2.2.2]octane-2-carboxylic acid;

(3R,4S,5S,6S)-5-(5-N-methyl-methanesulfonylamino-2-methoxybenzyl-amino)-6-diphenylmethyl-1-azabicyclo[2.2.2]octane-2-carboxylic acid;

(3R,4S,5S,6S)-5-(2-methoxy-5-methylsulfinylbenzyl-amino)-6-diphenylmethyl-1-azabicyclo[2.2.2]octane-2-carboxylic acid;

(3R,4S,5S,6S)-5-(2-methoxy-5-trifluoromethoxybenzyl-amino)-6-diphenylmethyl-1-azabicyclo[2.2.2]octane-2-carboxylic acid;

(3R,4S,5S,6S)-5-(2-methoxy-5-methylsulfonylbenzyl-amino)-6-diphenylmethy1-1-azabicyclo[2.2.2]octane-2-carboxylic acid; and

(3R,4S,5S,6S)-5-(5-dimethylamino-2-methoxybenzylamino)-6-diphenylmethyl-1-azabicyclo[2.2.2]octane-2-carboxylic acid;

and the pharmaceutically acceptable salts of the foregoing compounds.

The following references refer, collectively, to quinuclidine, piperidine, ethylene diamine, pyrrolidine and azanorbornane derivatives and related compounds that exhibit activity as NK-1 receptor antagonists which can be used in the pharmaceutical methods of this invention: U.S. Pat. No. 5,162,339, which issued on Nov. 11, 1992; U.S. Pat. No. 5,232,929, which issued on Aug. 3, 1993; World Patent Application WO 92/20676, published Nov. 26, 1992; World Patent Application WO 93/00331, published Jan. 7, 1993; World Patent Application WO 92/21677, published Dec. 10, 1992; World Patent Application WO 93/00330, published Jan. 7, 1993; World Patent Application WO 93/06099, published Apr. 1, 1993; World Patent Application WO 93/10073, published May 27, 1993; World Patent Application WO 92/06079, published Apr. 16, 1992, World Patent Application WO 92/12151, published Jul. 23, 1992; World Patent Application WO 92/15585, published Sep. 17, 1992; World Patent Application WO 93/10073, published May 27, 1993; World Patent Application WO 93/19064, published Sep. 30, 1993; World Patent Application WO 94/08997, published Apr. 28, 1994; World Patent Application WO 94/04496, published Mar. 3, 1994; World Patent Application WO 94/13663, published Jun. 23, 1994; World Patent Application WO 94/20500, published Sep. 15, 1994; World Patent Application PCT/IB94/00221, filed on Jul. 18, 1994; World Patent Application PCT/JP94/00781, filed on May 13, 1994; World Patent Application PCT/JP94/01092, filed on Jul. 5, 1994; and World Patent Application PCT/JP94/01514, filed on Sep. 13, 1994; U.S. patent application Ser. No. 988,653, filed Dec. 10, 1992; U.S. patent application Ser. No. 026,382, filed Mar. 4, 1993; U.S. patent application Ser. No. 123,306, filed Sep. 17, 1993, and U.S. patent application Ser. No. 072,629, filed Jun. 4, 1993. The foregoing patents and patent applications are incorporated herein by reference in their entirety.

Other NK-1 receptor antagonists that can be used in the pharmaceutical methods of this invention are those compounds and pharmaceutically acceptable salts described in the following references: European Patent Application EP 499,313, published Aug. 19, 1992; European Patent Application EP 520,555, published Dec. 30, 1992; European Patent Application EP 522,808, published Jan. 13, 1993, European Patent Application EP 528,495, published Feb. 24, 1993, PCT Patent Application WO 93/14084, published Jul. 22, 1993, PCT Patent Application WO 93/01169, published Jan. 21, 1993, PCT Patent Application WO 93/01165, published Jan. 21, 1993, PCT Patent Application WO 93/01159, published Jan. 21, 1993, PCT Patent Application WO 92/20661, published Nov. 26, 1992, European Patent Application EP 517,589, published Dec. 12, 1992, European Patent Application EP 428,434, published May 22, 1991, and European Patent Application EP 360,390, published Mar. 28, 1990; PCT Patent Application WO 95/04042, published Feb. 9, 1995, PCT Patent Application WO 95/08549, published Mar. 30, 1995, PCT Patent Application WO 95/19344, published Jul. 20, 1995, PCT Patent Application WO 95/23810, published Sep. 8, 1995, and PCT Patent Application WO 95/20575, published Aug. 3, 1995. These publications are also incorporated herein by reference in their entireties.

More preferably, the NK-1 receptor antagonist is a compound of Formula 1, Formula 2 or a pharmaceutically acceptable salt thereof:

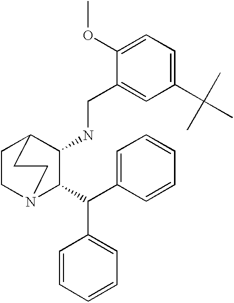

Formula 1 (2S.3S) (2-Benzhydryl-1-aza-bicyclo[2.2.2]oct-3-yl)-(5-tert-butyl-2-methoxy-benzyl)-amine

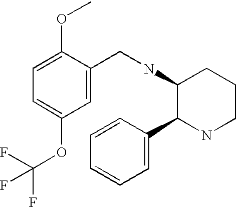

Formula 2 (2S,3S)(2-Methoxy-5-trifluoromethoxy-benzyl)-(2-phenyl-piperidin-3-yl)-amine)

The NK-1 receptor antagonists used in the present invention may have chiral centers and therefore exist in different enantiomeric forms. This invention relates to uses for all optical isomers and all stereoisomers of compounds of the Formula 1or Formula 2 and mixtures thereof.

The NK-1 receptor antagonist should be chosen from CNS-penetrant NK-1 receptor antagonists. It is well known to those in the art how to determine if an NK-1 receptor antagonist is CNS penetrant. For example, tests are disclosed in WO 98/15277.

NK-1 receptor antagonist includes compounds that have an NK-1 receptor affinity (IC₅₀) of less than 100 nM. Preferably, the NK-1 receptor antagonist has IC₅₀#10 nM, and more preferably IC₅₀# 1 nM.

To determine NK-1 receptor affinity, one of the NK-1 receptor binding assays well known in the art may be used. One such assay is described by Cascieri et al., J. Pharmacol. Exp. Ther.,1992, 42, 458.

It is understood that the amino acid sequence of the NK-1 receptor may differ between species. Accordingly, the assay for NK-1 receptor binding preferably involves an NK-1 receptor naturally occurring in the species of companion animal to be treated. However, it is within the skill of one in the art to determine if the binding results from an assay in which an NK-1 receptor from a different species is used are sufficient to predict with reasonable certainty NK-1 receptor binding in the species to be treated.

The NK-1 receptor antagonists can be administered via oral, parenteral, inhalation or topical routes, preferably orally.

To determine an efficacious dosage, multiple complete cross-over studies can be performed with the NK-1 receptor antagonist in the species of companion animal to be treated at various doses. The optimal dose is selected based on the maximal ability to decrease the time spent in abnormal behaviors.

For example, non-peptidyl NK-1 receptor antagonists are most desirably administered in dosages ranging from about 0.01 mg/kg animal body weight to about 5 mg/kg animal body weight per dosage, preferably in dosages of from about 0.1 mg/kg to 0.3 mg/kg. The dosage is administered from once to six times per day, and preferably is administered once or twice a day. Peptidyl NK-1 receptor antagonists are preferably administered parenterally or through inhalation, in dosages readily determinable by those of skill in the art.

Duration of therapy may vary depending on the animal's condition. Duration of therapy can be for two to four months when administered concurrent with behavioral therapy to reduce or eliminate the abnormal anxiety behavior. After acceptable alternative behavior is maintained for four to six weeks, the animal can be weaned off of the medication. In some instances, lifelong medication may be needed to maintain acceptable behavior. Administration should occur at least until the abnormal behavior is reduced to an acceptable level.

Dosages can be determined by dose titration as is known to those skilled in the art. An example of a dose titration for (2S.3S)(2-Benzhydryl-1-aza-bicyclo[2.2.2]oct-3-yl)-(5-tert-butyl-2-methoxy-benzyl)-amine is provided in the examples that follow.

Variations may occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

The NK-1 receptor antagonists used in the invention may be administered alone or in combination with pharmaceutically acceptable carriers or diluents by any of the routes previously indicated, and such administration may be carried out in single or multiple doses. More particularly, the novel therapeutic agents of this invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the therapeutically-effective compounds of this invention are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a therapeutic compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

The method of treatment of the present invention also may be combined with other treatment therapies, and particularly with other therapies directed to treating abnormal aggressive behaviors or abnormal anxiety behaviors. For example, the administration of an NK-1 receptor antagonist to a companion animal to treat abnormal anxiety behaviors may be combined with the administration of tricyclic antidepressants such as clomipramine and amitryptyline, the administration of agents that act as seritonin, norepinephrine and/or dopamine reuptake inhibitors, including venlafaxine; sedative agents such as benzodiazepenes (including Diazepam®) and phenothiazines (including Acepromazine®), and/or the administration of selective seritonin reuptake inhibitors, such as fluoxetine hydrochloride (sold under the tradename Prozac®) and sertraline hydrochloride (sold under the tradename Zoloft®).

Preferably, the methods of treatment of the present invention are combined with concurrent behavior modification training. An example of one behavior modification training is desensitization by exposing the animal to arousing stimuli at a level that does not evoke a response and then rewarding the animal for staying calm.

Canine Anxiety Model:

The present invention also concerns a method for testing compounds for anxiolytic effect in dogs. The model is designed to induce separation anxiety as well as anxiety due to novel visual and auditory stimuli. To determine whether a test compound has an anxiolytic effect in dogs, separation anxiety and anxiety associated with novel sight and sound stimuli are measured both with and without administration of the test compound.

In one preferred embodiment, observations are made on five different anxiety behaviors during a 15-minute separation phase and a 15-minute stimulation phase. These anxiety behaviors are vocalization (barking, crying, growling); hyperactivity (jumping, pacing, circling); destruction (chewing, pawing, digging); salivation; and tremor. The duration of time (in seconds) that each behavior is exhibited is recorded and totaled at the end of each 15-minute phase. In addition to direct observation, dogs are videotaped during testing for documentation of behaviors.

In one preferred embodiment, a dog is removed from a group pen and placed in an isolation cage. A tester, positioned behind a screen out of view, starts a timer for 15 minutes and a video recorder. The duration of time (in seconds) an individual animal exhibits each behavior is recorded. At the end of the separation phase, the timer is re-set to 15 minutes and a child-size doll mounted on a remote controlled car is driven around the pen continuously. After 10 minutes, the doll and car are driven behind the screen and a child's fazer gun with a variety of sounds is continually activated for 5 minutes. At the end of the stimulation phase, the accumulated time for each behavior is recorded and the video recorder stopped. The dog is removed from the isolation cage and returned to the group pen.

Preferably, a complete cross-over study design with test compound and placebo is used. Each treatment period is 21 days in length. Study “day 0” is used to designate the initial day of dosing. Anxiety testing is conducted on study days 6 and 20 within each period. Animals are allocated to treatment group, sequence, pen and evaluation order using a randomization plan. Dogs remain within the same sequence during progression of the cross-over phases of the study so that all possible treatment orders are evenly represented. Preferably, “day 0” is staggered such that 3-6 dogs are evaluated per day. Treatment administration is adjusted on testing days such that each animal is tested at the predicted Cmax for the test compound. Anxiety testing is preferably conducted between 11 AM and 4 PM to account for time-of-day variations in behavior. Animals are fed at the conclusion of testing. A three-week washout is observed between periods.

Selection of Study Candidates:

Preferably, dogs that have previously displayed symptoms of separation anxiety are used in this model. A screening model is used in the selection of study candidates. For example, a dog is removed from a group pen and placed alone in a cage in an adjacent room. An observer stands behind a one-way glass and observes the dog for the following behaviors: excessive vocalization (barking, crying, howling), hyperactivity (pacing, circling, jumping) and destructive behaviors (digging, chewing, pawing). The dog is observed for 10 minutes. If no anxious behavior is observed, the dog is returned to the pool. Dogs exhibiting anxious behaviors are screened once weekly for 4 weeks. If the behaviors are repeated and consistent, the dog moves on to the next selection period. The dog is challenged in the separation phase of the canine anxiety model once weekly for six weeks. Preferably, the study only uses dogs that have exhibited anxiety each week during the six weeks of screening.

It is envisioned that the method of testing compounds for anxiolytic behavior in dogs may be altered from the preferred embodiment as would be evident to one skilled in the art. For example, the separation period of a fixed duration during which behaviors are observed may be fifteen minutes, as described in the preferred embodiment, or may be any other time period of or predetermined duration that allows sufficient time to observe behaviors such that distinctions may be drawn between behavior with or without administration of the test compound.

The present invention is illustrated by the following examples. It will be understood, however, that the invention is not limited to the specific details of these examples.

### EXAMPLE 1

To evaluate the anxiolytic effects of a compound of Formula 1 vs. placebo after seven and twenty-one days of parenteral dosing at 0.1 mg/kg SID, the following procedure was followed.

Test Materials:

| Compound of Formula 1 | | Placebo | |
|---|---|---|---|
| Dosage form | Subcutaneous Injection | Dosage form | Subcutaneous |
| Injection | | | |
| Potency | 69% | Potency | 0% |
| Formulation | Dissolved in 20% | Formulation | 20% (w/v) |
| | (w/v) SBE | | |
| | SBE cyclodextrin | | |
| | in water to make a | | |
| | cyclodextrin in | | |
| | base equivalent | | water |
| | solution in water | | |
| | of 5 mg/ml | | |

Study Design:

Adult dogs with spontaneous anxiety to isolation and/or unfamiliar humans were treated for twenty-one consecutive days with the compound of Formula 1 or a placebo control. Dogs were tested after seven and twenty-one days of dosing. “Day 0” was used to identify the initial day of dosing. The study was conducted using a double-blind crossover design. Treatment periods were 21 days in duration, with a 28 day washout period between treatments. Dogs were randomly assigned to treatment groups such that those receiving compound at the first replicate received placebo on the second replicate. The placebo treatments served as the negative control.

All study participants were blinded to treatment groups. The compound of Formula 1 was administered at a dose of 0.1 mg/kg. The volume of vehicle control used for the placebo treatments was equivalent to the calculated volume of test compound that would have been administered had the animal been receiving drug. All test articles were administered by subcutaneous injection once daily.

Behavior Analysis:

On the day of testing, each dog was placed in a cage in an isolated room for 15 minutes. A hidden observer timed any anxious behavior. Animals were tested on dosing day 6 and day 20, starting 2 hours after the dose of the day.

Abnormal anxiety behaviors were studied. Observations were made on two different anxiety behaviors during a 15-minute separation phase. These behaviors are vocalization (barking, crying, growling) and hyperactivity (jumping, pacing, circling). The duration of time (in seconds) that each behavior was exhibited was measured using a remote data capture device and totaled for each animal at the end of each 15 minute period.

Results:

The following table (Table 1) summarizes the mean data from dogs included in the experiment.

TABLE 1
The Compound of Formula 1 @ 0.1 mg/kg Vs. Placebo
Geometric Mean Time (seconds)
Separation Phase

| | | Vocalization | Hyperactivity |
|---|---|---|---|
| Treatment | Day of Study | Mean | Mean |
| Placebo | Day 6 | 36.1 | 25.8 |
| Formula 2 | Day 6 | 29.0 | 24.1 |
| Placebo | Day 20 | 53.1 | 42.5 |
| Formula 2 | Day 20 | 25.5 | 19.1 |

As is evident from the data in Table 1, the NK-1 receptor antagonist of Formula 1 is more effective than placebo at treating vocalization and hyperactivity resulting from separation.

### EXAMPLE2

Various dosages of the compound of Formula 1 were tested to determine an optimal dose. Anxiety associated with separation and with novel sight and sound stimuli was measured.

Observations are made on two different anxiety behaviors during a 15-minute separation phase and a 15-minute stimulation phase. These behaviors are vocalization (barking, crying, growling) and hyperactivity (jumping, pacing, circling). The duration of time (in seconds) that each behavior is exhibited is recorded and totaled at the end of each 15-minute phase. In addition to direct observation, dogs are videotaped during testing for documentation of behaviors.

The dog is removed from a group pen and placed in an isolation cage in an adjacent room. The tester, positioned behind a screen out of view, starts a timer for 15 minutes and the video recorder. The duration of time (in seconds) that an individual animal exhibits each behavior is recorded. At the end of the separation phase, the timer is re-set to 15 minutes and a child-size doll mounted on a remote controlled car is driven around the pen continuously. After 10 minutes, the doll and car are driven behind the screen and a child's fazer gun with a variety of sounds is continually activated for 5 minutes. At the end of the stimulation phase, the accumulated time for each behavior is recorded and the video recorder stopped. The dog is removed from the isolation cage and returned to the group pen.

TABLE 2
Compound of Formula 1 @ 1 mg/kg Vs. Placebo
Geometric Mean Time (second)
“Comp. 1” is a Compound of Formula 1

| | | | |
|---|---|---|---|
| | | Vocalization | Hyperactivity |
| Treatment | Study Day | Mean | Mean |

Separation Phase

| | | | |
|---|---|---|---|
| Comp. 1 | Day 6 | 5.8 | 23.3 |
| Placebo | Day 6 | 7.9 | 15.0 |
| Comp. 1 | Day 20 | 6.9 | 16.8 |
| Placebo | Day 20 | 8.5 | 17.0 |

Stimulation Phase

| | | | |
|---|---|---|---|
| Comp. 1 | Day 6 | 6.5 | 22.7 |
| Placebo | Day 6 | 8.9 | 28.8 |
| Comp. 1 | Day 20 | 7.4 | 36.0 |
| Placebo | Day 20 | 7.3 | 20.9 |

TABLE 3
Compound of Formula 1 @ 0.3 mg/kg Vs. Day −1
Geometric Mean Time (second)

| | | Vocalization | Hyperactivity |
|---|---|---|---|
| Phase | Study Day | Mean | Mean |
| Separation | Day −1 | 31.8 | 71.4 |
| Separation | Day 6 | 20.9 | 49.7 |
| Separation | Day 20 | 18.2 | 39.1 |
| Stimulation | Day −1 | 33.4 | 104.9 |
| Stimulation | Day 6 | 15.9 | 43.1 |
| Stimulation | Day 20 | 19.2 | 41.9 |

TABLE 4
Compound of Formula 1 @ 0.1 mg/kg Vs. Day −1
Geometric Mean Time (second)

| | | Vocalization | Hyperactivity |
|---|---|---|---|
| Phase | Study Day | Mean | Mean |
| Separation | Day −1 | 35.9 | 47.2 |
| Separation | Day 6 | 15.5 | 24.7 |
| Separation | Day 20 | 9.1 | 14.8 |
| Stimulation | Day −1 | 48.9 | 70.9 |
| Stimulation | Day 6 | 36.8 | 48.8 |
| Stimulation | Day 20 | 24.7 | 28.4 |

TABLE 5
Compound of Formula 1 @ 0.03 mg/kg Vs. Day −1
Geometric Mean Time (second)

| | | | |
|---|---|---|---|
| | | Vocalization | Hyperactivity |
| Phase | Study Day | Mean | Mean |

| | | | |
|---|---|---|---|
| Separation | Day −1 | 22.1 | 46.0 |
| Separation | Day 6 | 10.6 | 19.4 |
| Separation | Day 20 | 12.1 | 20.1 |
| Stimulation | Day −1 | 38.6 | 54.9 |
| Stimulation | Day 6 | 24.5 | 38.6 |
| Stimulation | Day 20 | 35.6 | 28.2 |

TABLE 6
Compound of Formula 1 @ 0.1 mg/kg Vs. Placebo
Geometric Mean Time (second)

| | |
|---|---|
| Vocalization | Hyperactivity |

| Treatment | Study Day | Mean | Mean |
|---|---|---|---|
| Separation Phase | | | |
| Comp. 1 | Day 6 | 29.0 | 24.1 |
| Placebo | Day 6 | 36.1 | 25.8 |
| Comp. 1 | Day 20 | 25.5 | 19.1 |
| Placebo | Day 20 | 53.1 | 42.5 |
| Stimulation Phase | | | |
| Comp. 1 | Day 6 | 35.3 | 46.7 |
| Placebo | Day 6 | 46.7 | 47.2 |
| Comp. 1 | Day 20 | 26.4 | 51.2 |
| Placebo | Day 20 | 56.2 | 69.6 |

Tables 3-5 refer to “Day -1” which is the day prior to initial dosage. The data shows that among those dosages tested, the most efficacious dosage in dogs of the compound of Formula 1 is 0.1 mg/kg SC.

### EXAMPLE 3

Adult dogs received ten consecutive oral doses of a compound of Formula 2 at 5 mg/kg BID or placebo in a cross-over design and effects on behavior were determined beginning one-hour post-dose.

| Materials: | |
|---|---|
| Compound | Compound of Formula 2 |
| Dosage form | Oral capsule |
| Potency | capsules at 5 mg/kg activity |
| Management: | Water: ad libitum |
| | Feed: Standard high energy canine ration |
| | Feed at 1300, remove food at 1500 |

Methods:

Preparation of Dose:

Appropriate amounts of the compound of Formula 2 were weighed and placed in capsules. Capsules were back-filled with dextrose.

Administration of Dose:

Capsules were administered by placing them in the back of the throat and allowing the dogs to swallow. On the day of behavior testing, dogs were fasting at dosing.

Design:

Dogs were divided equally into two treatment groups. Treatment Group 1 received 10 consecutive oral doses of the compound of Formula 2 BID at 5 mg/kg and Treatment Group 2 received 10 consecutive oral dextrose placebos BID. At the conclusion of the first half of the study, the protocol was repeated and dogs in Treatment Group 1 received 10 consecutive oral dextrose placebos BID and dogs in Treatment Group 2 received 10 consecutive oral doses of the compound of Formula 2 BID at 5 mg/kg.

Behavior Assessment:

Dogs began compound dosing at 1830 and continued for 10 consecutive BID doses. The final dose (dose #10) occurred the morning of testing, one hour prior to the start. Dogs were tested in three 15-minute testing phases in which behavior was scored every 5 minutes. Dogs were videotaped during the testing period.

TABLE 7
Effect of Multi-dose compound of Formula 2 @ 5 mg/kg
on Behavior Scores* in Dogs
*Estimated duration of time in seconds

| | |
|---|---|
| Separation | Separation |
| Vocalization 0-15 min | Hyperactivity 0-15 min |

| Dog ID | Placebo | Comp. 2 | Placebo | Comp. 2 |
|---|---|---|---|---|
| 219622 | 42 | 22 | 50 | 30 |
| 70954 | 2 | 0 | 4 | 2 |
| 234923 | 6 | 0 | 6 | 2 |
| HIHMFU | 0 | 0 | 50 | 12 |
| 240893 | 4 | 0 | 6 | 6 |
| 215155 | 14 | 12 | 6 | 2 |
| 227641 | 6 | 2 | 2 | 2 |
| 2943450 | 90 | 30 | 36 | 18 |
| Mean | 20.5 | 8.25 | 20.0 | 9.25 |

Score System:
Converted original score of 1,2,3 to estimated time in seconds
1 = 2 seconds
2 = 10 seconds
3 = 30 seconds

1. A method of treating abnormal anxiety behavior in companion animals comprising administering to a companion animal in need thereof a therapeutically effective amount of an NK-1 receptor antagonist.

2. The method of claim 1, wherein the abnormal anxiety behavior is selected from the group consisting of vocalization, hyperactivity, destruction, abnormal sleep, abnormal feeding, abnormal drinking, abnormal grooming, abnormal elimination, abnormal fears and phobias, and socialization disorders.

3. The method of claim 2, wherein the abnormal anxiety behavior is selected from the group consisting of vocalization, hyperactivity, destruction, abnormal feeding, and abnormal elimination.

4. The method of claim 3, wherein the abnormal anxiety behavior is selected from the group consisting of vocalization, hyperactivity, and destruction.

5. The method of claim 4, wherein the abnormal anxiety behavior is vocalization.

6. The method of claim 4, wherein the abnormal anxiety behavior is hyperactivity.

7. The method of claim 4, wherein the abnormal anxiety behavior is destruction.

8. The method of claim 1, wherein the companion animal is selected from the group consisting of dogs, cats, and horses.

9. The method of claim 8, wherein the companion animal is a dog.

10. The method of claim 8 wherein the companion animal is a cat.

11. The method of claim 8, wherein the companion animal is a horse.

12. The method of claim 1, wherein the NK-1 receptor antagonist is selected from the group consisting of:
(2S,3S)-3-(5-tert-butyl-2-methoxybenzyl)amino-2-(3-trifluoromethoxyphenyl)piperidine;
(2S,3S)-3-(2-isopropoxy-5-trifluoromethoxybenzyl)amino-2-phenyl-piperidine;
(2S,3S)-3-(2-ethoxy-5-trifluoromethoxybenzyl)amino-2-phenyl-piperidine;
(2S,3S)-3-(2-methoxy-5-trifluoromethoxybenzyl)-amino-2-phenylpiperidine;
(2S,3S)-3(-5-tert-butyl-2-trifluoromethoxybenzyl)amino-2-phenylpiperidine;
2-(diphenylmethyl)-N-(2-methoxy-5-trifluoromethoxy-phenyl)methyl-1-azabicyclo[2.2.2]octan-3-amine;
(2S,3S)-3-[5-chloro-2-(2,2,2-trifluoroethoxy)-benzyl]amino-2-phenylpiperidine;
(2S,3S)-3-(5-tert-butyl-2-trifluoromethoxybenzyl)amino-2-phenylpiperidine;
(2S,3S)-3-(2-isopropoxy-5-trifluoromethoxybenzyl)amino-2-phenylpiperidine;
(2S,3S)-3-(2-difluoromethoxy-5-trifluoromethoxybenzyl)-amino-2-phenylpiperidine;
(2S,3S)-2-phenyl-3-[2-(2,2,2-trifluoroethoxybenzyl)-aminopiperidine;
(2S,3S)-2-phenyl-3-(2-trifluoromethoxybenzyl)]aminopi-peridine;
cis-3-(2-chlorobenzylamino)-2-phenylpiperidine;
cis-3-(2-trifluoromethylbenzylamino)-2-phenyl-piperidine;
cis-3-(2-methoxybenzylamino)-2-(2-fluorophenyl)-piperidine;
cis-3-(2-methoxybenzylamino)-2-(2-chlorophenyl)-piperidine;
cis-3-(2-methoxybenzylamino)-2-(2-methylphenyl)-piperidine;
cis-3-(2-methoxybenzylamino)-2-(3-methoxyphenyl)-piperidine;
cis-3-(2-methoxybenzylamino)-2-(3-fluorophenyl)-piperidine;
cis-3-(2-methoxybenzylamino)-2-(3-chlorophenyl)-piperidine;
cis-3-(2-methoxybenzylamino)-2-phenylpiperidine;
cis-3-(2-methoxybenzylamino)-2-(3-methylphenyl)-piperidine;
cis-3-(2-methoxybenzylamino)-2-(4-fluorophenyl)-piperidine;
cis-3-(2-methoxybenzylamino)-2-(3-thienyl)-piperidine;
cis-3-(2-methoxybenzylamino)-2-phenylazacyclo-heptane;
3-(2-methoxybenzylamino)-4-methyl-2-phenylpiperidine;
3-(2-methoxybenzylamino)-5-methyl-2-phenylpiperidine;
3-(2-methoxybenzylamino)-6-methyl-2-phenylpiperidine;
(2S,3S)-3-(2-methoxybenzylamino)-2-phenylpiperidine;
(2S,3S)-1-(5-carboethoxypent-1-yl)-3-(2-methoxybenzyl-amino)-2-phenylpiperidine;
(2S,3S)-1-(6-hydroxy-hex-1-yl)-3-(2-methoxybenzyl-amino)-2-phenylpiperidine;
(2S,3S)-1-(4-hydroxy-4-phenylbut-1-yl)-3-(2-methoxy-benzylamino)-2-phenylpiperidine;
(2S,3S)-1-(4-oxo-4-phenylbut-1-yl)-3-(2-methoxybenzyl-amino)-2-phenylpiperidine;
(2S,3S)-1-(5,6-dihydroxyhex-1-yl)-3-(2-methoxybenzyl-amino)-2-phenylpiperidine;
cis-3-(5-fluoro-2-methoxybenzylamino)-2-phenyl-piperidine;
(2S,3S)-1-[4-(4-fluorophenyl)-4-oxobut-1-yl]-3-(2-methoxybenzylamino)-2-phenylpiperidine;
(2S,3S)-1-[4-[4-fluorophenyl)-4-hydroxybut-1-yl]-3-(2-methoxybenzylamino)-2-phenylpiperidine;
cis-3-(2-methoxy-5-methylbenzylamino)-2-phenyl-piperidine;
(2S,3S)-1-(4-benzamidobut-1-yl)-3-(2-methoxybenzyl-amino)-2-phenylpiperidine;
cis-3-(2-methoxynaphth-1-ylmethylamino)-2-phenyl-piperidine;
(2S,3S)-3-(2-methoxybenzylamino)-1-(5-N-methyl-carboxamidopent-1-yl)-2-phenylpiperidine;
(2S,3S)-1-(4-cyanobut-1-yl)-3-(2-methoxybenzylamino)-2-phenylpiperidine;
(2S,3S)-1-[4-(2-naphthamido)but-1-yl]-3-(2-methoxy-benzylamino)-2-phenylpiperidine;
(2S,3S)-1-(5-benzamidopent-1-yl)-3-(2-methoxybenzyl-amino)-2-phenylpiperidine;
(2S,3S)-1-(5-aminopent-1-yl)-3-(2-methoxybenzylamino)-2-phenylpiperidine;
(2S,3S)-3-(5-chloro-2-methoxybenzylamino)-2-phenyl-piperidine;
(2S,3S)-3-(2,5-dimethoxybenzylamino)-2-phenyl-piperidine;
cis-3-(3,5-difluoro-2-methoxybenzylamino)-2-phenyl-piperidine;
cis-3-(4,5-difluoro-2-methoxybenzylamino)-2-phenyl-piperidine;
cis-3-(2,5-dimethoxybenzylamino)-1-[4-(4-fluorophenyl)-4-oxobut-1-yl]-2-phenylpiperidine;
cis-3-(5-chloro-2-methoxybenzylamino)-1-(5,6-dihydroxyhex-1-yl)-2-phenylpiperidine;
cis-1-(5,6-dihydroxyhex-1-yl)-3-(2,5-dimethoxy-benzylamino)-2-phenylpiperidine;
cis-2-phenyl-3-[-2(prop-2-yloxy)benzylamino]piperidine;
cis-3-(2,5-dimethoxybenzyl)amino-2-(3-methoxy-phenyl)piperidine hydro-chloride;
cis-3-(5-chloro-2-methoxybenzyl)amino-2-(3-methoxy-phenyl)piperidine dihydrochloride;
cis-3-(5-chloro-2-methoxybenzyl)amino-2-(3-chloro-phenyl)piperidine dihydrochloride;3-(2-methoxybenzylamino)-2,4-diphenylpiperidine;
cis-3-(2-methoxybenzylamino)-2-phenylpyrrolidine;
(2S,3S)-3-(5-ethyl-2-methoxybenzyl)amino-2-phenyl-piperidine;
(2S,3S)-3-(5-n-butyl-2-methoxybenzyl)amino-2-phenyl-piperidine;
(2S,3S)-3-(2-methoxy-5-n-propylbenzyl)amino-2-phenyl-piperidine;
(2S,3S)-3-(5-isopropyl-2-methoxybenzyl)amino-2-phenyl-piperidine;
(2S,3S)-3-(5-s-butyl-2-methoxybenzyl)amino-2-phenyl-piperidine;
(2S,3S)-3-(5-t-butyl-2-methoxybenzyl)amino-2-phenyl-piperidine;
(2S,3S)-3-(2-methoxy-5-phenylbenzyl)amino-2-phenyl-piperidine;
2,4-dimethylthiazole-5-sulfonic acid [4-methoxy-3-((2S,3S)-2-phenylpiperidin-3-ylaminomethyl)phenyl]-methylamide;
N-(4,5-dimethylthiazol-2-yl)-N-[4-methoxy-3-((2S,3S)-2-phenylpiperidin-3-yl-aminomethyl)phenyl]-methanesulfonamide;
{5-[(4,5-dimethylthiazol-2-yl)methylamino]-2-methoxybenzyl}-((2S,3S)-2-phenylpiperidin-3-yl)amine;
{5-(4,5-dimethylthiazol-2-ylamino)-2-methoxybenzyl}-((2S,3S)-2-phenylpiperidin-3-ylamine;
4,5-dimethylthiazole-2-sulfonic acid methyl-[3-((2S,3S)-2-phenylpiperidin-3-ylamino-methyl)-4-trifluoromethoxyphenyl]-amide;
2,4-dimethylthiazole-5-sulfonic acid [4-isopropoxy-3-((2S,3S)-2-phenylpiperidin-3-ylaminomethyl)phenyl]-methylamide;
2,4-dimethylthiazole-5-sulfonic acid [4-isopropoxy-3-((2S,3S)-2-phenylpiperidin-3-yl-aminomethyl)phenyl]-isopropylamide;
2,4-dimethylthiazole-5-sulfonic acid [4-methoxy-3-((2S,3S)-2-phenylpiperidin-3-ylamino-methyl)phenyl]-isopropylamide;
2,4-dimethylthiazole-5-sulfonic acid [4-methoxy-3-((2S,3S)-2-phenylpiperidin-3-ylaminomethyl)phenyl]-isobutylamide;
2,4-dimethylthiazole-5-sulfonic acid [4-isopropoxy-3-((2S,3S)-2-phenylpiperidin-3-ylaminomethyl)phenyl]-isobutylamide;
(2S,3S)-N-(5-isopropyl-2-methoxyphenyl)methyl-2-diphenylmethyl-1-azabicyclo-[2.2.2]octan-3-amine;
(2S,3S)-N-(5-tert-butyl-2-methoxyphenyl)methyl-2-diphenylmethyl-1-azabicyclo[2.2.2]octan-3-amine;
(2S,3S)-N-(5-methyl-2-methoxyphenyl)methyl-2-diphenylmethyl-1-azabicyclo[2.2.2]octan-3-amine;
(2S,3S)-N-(5-ethyl-2-methoxyphenyl)methyl-2-diphenylmethyl-1-azabicyclo[2.2.2]octan-3-amine;
(2S,3S)-N-(5-isopropyl-2-methoxyphenyl)methyl-2-diphenylmethyl-1-azabicyclo[2.2.2]octan-3-amine;
(2S,3S)-N-(5-sec-butyl-2-methoxyphenyl)methyl-2-diphenylmethyl-1-azabicyclo[2.2.2]octan-3-amine;
(2S,3S)-N-(5-n-propyl-2-methoxyphenyl)methyl-2-diphenylmethyl-1-azabicyclo[2.2.2]octan-3-amine;
(3R,4S,5S,6S)-N,N-diethyl-5-(5-isopropyl-2-methoxy-benzylamino)-6-diphenylmethyl-1-azabicyclo[2.2.2]octane-3-carboxamide;
(3R,4S,5S,6S)-N,N-diethyl-5-(2,5-dimethoxybenzylamino)-6-diphenylmethyl-1-azabicyclo[2.2.2]octane-3-carboxamide;
(3R,4S,5S,6S)-5-(5-isopropyl-2-methoxybenzylamino)-6-diphenylmethyl-1-azabicyclo[2.2.2]octane-3-carboxylic acid;
(3R,4S,5S,6S)-5-(2-methoxy-2-methylthiobenzylamino)-6-diphenylmethyl-1-azabicyclo[2.2.2]octane-3-carboxylic acid;
(3R,4S,5S,6S)-5-(2,5-dimethoxybenzylamino)-6-diphenyl-methyl-1-azabicyclo-[2.2.2]octane-3-carboxylic acid;
(3R,4S,5S,6S)-5-(2-methoxy-5-methylbenzylamino)-6-diphenylmethyl-1-azabicyclo[2.2.2]octane-3-carboxylic acid;
(3R,4S,5S,6S)-5-(5-ethyl-2-methoxybenzylamino)-6-diphenylmethyl-1-azabicyclo[2.2.2]octane-3-carboxylic acid;
(3R,4S,5S,6S)-5-(2-methoxyl-5-n-propylbenzylamino)-6-diphenylmethyl-1-azabicyclo[2.2.2]octane-3-carboxylic acid;
(3R,4S,5S,6S)-5-(5-sec-butyl-2-methoxybenzylamino)-6-diphenylmethyl-1-azabicyclo[2.2.2]octane-3-carboxylic acid;
(3R,4S,5S,6S)-5-(5-N-methyl-methanesulfonylamino-2-methoxy-benzylamino)-6-diphenylmethyl-1-azabicyclo[2.2.2]octane-3-carboxylic acid;
(3R,4S,5S,6S)-5-(2-methoxy-5-methylsulfinylbenzyl-amino)-6-diphenylmethyl-1-azabicyclo[2.2.2]octane-3-carboxylic acid;
(3R,4S,5S,6S)-5-(2-methoxy-5-trifluoromethoxybenzyl-amino)-6-diphenylmethyl-1-azabicyclo[2.2.2]octane-3-carboxylic acid;
(3R,4S,5S,6S)-5-(2-methoxy-5-methylsulfonylbenzyl-amino)-6-diphenylmethyl-1-azabicyclo[2.2.2]octane-3-carboxylic acid;
(3R,4S,5S,6S)-5-(5-dimethylamino-2-methoxybenzylamino)-6-diphenylmethyl-1-azabicyclo[2.2.2]octane-3-carboxylic acid;
(3R,4S,5S,6S)-5-(5-isopropyl-2-methoxybenzylamino)-6-diphenylmethyl-1-azabicyclo[2.2.2]octane-2-carboxylic acid;
(3R,4S,5S,6S)-5-(2-methoxy-5-methylthiobenzylamino)-6-diphenylmethyl-1-azabicyclo[2.2.2]octane-2-carboxylic acid;
(3R,4S,5S,6S)-5-(2,5-dimethoxy benzylamino)-6-diphenylmethyl-1-azabicyclo[2.2.2]octane-2-carboxylic acid;
(3R,4S,5S,6S)-5-(2-methoxy-5-methylbenzylamino)-6-diphenylmethyl-1-azabicyclo[2.2.2]octane-2-carboxylic acid;
(3R,4S,5S,6S)-5-(5-ethyl-2-methoxy benzylamino)-6-diphenylmethyl-1-azabicyclo[2.2.2]octane-2-carboxylic acid;
(3R,4S,5S,6S)-5-(2-methoxyl-5-n-propylbenzylamino)-6-diphenylmethyl-1-azabicyclo[2.2.2]octane-2-carboxylic acid;
(3R,4S,5S,6S)-5-(5-sec-butyl-2-methoxybenzylamino)-6-diphenylmethyl-1-azabicyclo[2.2.2]octane-2-carboxylic acid;
(3R,4S,5S,6S)-5-(5-N-methyl-methanesulfonylamino-2-methoxybenzyl-amino)-6-diphenylmethyl-1-azabicyclo[2.2.2]octane-2-carboxylic acid;
(3R,4S,5S,6S)-5-(2-methoxy-5- methylsulfinylbenzyl-amino)-6-diphenylmethyl-1-azabicyclo[2.2.2]octane-2-carboxylic acid;
(3R,4S,5S,6S)-5-(2-methoxy-5-trifluoromethoxybenzyl-amino)-6-diphenylmethyl-1-azabicyclo[2.2.2]octane-2-carboxylic acid;
(3R,4S,5S,6S)-5-(2-methoxy-5-methylsulfonylbenzyl-amino)-6-diphenylmethyl-1-azabicyclo[2.2.2]octane-2-carboxylic acid; and
(3R,4S,5S,6S)-5-(5-dimethylamino-2-methoxybenzylamino)-6-diphenylmethyl-1-azabicyclo[2.2.2]octane-2-carboxylic acid; or pharmaceutically acceptable salts thereof.

13. The method of claim 1, wherein the NK-1 receptor antagonist is a compound of Formula 1, Formula 2, or a pharmaceutically acceptable salt thereof:

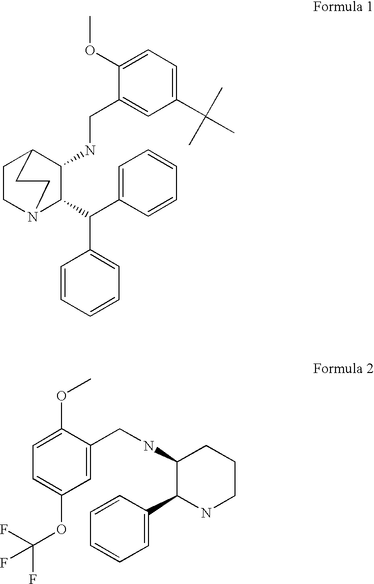

14. The use of an NK-1 receptor antagonist to prepare a medicinal composition to treat abnormal anxiety behavior in companion animals.

15. A method of screening a test compound to determine anxiolytic activity in dogs comprising (a) selecting a dog exhibiting an anxiety behavior; (b) administering the test compound to the dog; (c) separating the dog from views of other dogs and of humans; (d) measuring a first duration of time, the first duration of time being the time within a separation period of a fixed duration during which the anxiety behavior is exhibited; and (e) comparing the first duration of time with a second duration of time, wherein the second duration of time is the time within a separation period of the fixed duration that the anxiety behavior is exhibited in the dog when the dog has not received the test compound for at least forty-eight hours; wherein if the first duration of time is less than the second duration of time, the test compound is determined to have anxiolytic activity.

16. The method of claim 15 wherein the anxiety behavior is selected from the group consisting of vocalization, hyperactivity, destruction, abnormal sleep, abnormal feeding, abnormal drinking, abnormal grooming, abnormal elimination, abnormal fears and phobias, and socialization disorders.

17. The method of claim 16 wherein the anxiety behavior is selected from the group consisting of vocalization, hyperactivity, destruction, abnormal feeding, and abnormal elimination.

18. The method of claim 17 wherein the anxiety behavior is selected from the group consisting of vocalization, hyperactivity, and destruction.

19. The method of claim 15 wherein the second duration of time is the time within a separation period of the fixed duration that the anxiety behavior is exhibited in the dog when the dog has not received the test compound for at least three weeks.

20. A method of treating abnormal anxiety behavior in a companion animal, the method comprising evaluating the companion animal for the exhibition of an abnormal anxiety behavior, determining that the companion animal exhibits the abnormal anxiety behavior and thus is in need of treatment, and administering to the companion animal a therapeutically effective amount of an NK-1 receptor antagonist for a time sufficient to reduce or eliminate the abnormal anxiety behavior.

21. The method of claim 20, wherein the abnormal anxiety behavior is selected from the group consisting of vocalization, hyperactivity, destruction, abnormal sleep, abnormal feeding, abnormal drinking, abnormal grooming, abnormal elimination, abnormal fears and phobias, and socialization disorders.

22. The method of claim 21, wherein the abnormal anxiety behavior is selected from the group consisting of vocalization, hyperactivity, destruction, abnormal feeding, and abnormal elimination.

23. The method of claim 22, wherein the abnormal anxiety behavior is selected from the group consisting of vocalization, hyperactivity, and destruction.

24. The method of claim 23, wherein the abnormal anxiety behavior is vocalization.

25. The method of claim 23, wherein the abnormal anxiety behavior is hyperactivity.

26. The method of claim 23, wherein the abnormal anxiety behavior is destruction.

27. The method of claim 20, wherein the companion animal is selected from the group consisting of dogs, cats, and horses.

28. The method of claim 27, wherein the companion animal is a dog.

29. The method of claim 27, wherein the companion animal is a cat.

30. The method of claim 27, wherein the companion animal is a horse.

31. The method of claim 20, wherein the NK-1 receptor antagonist is selected from the group consisting of:
(2S,3S)-3-(5-tert-butyl-2-methoxybenzyl)amino-2-(3-trifluoromethoxyphenyl)piperidine;
(2S,3S)-3-(2-isopropoxy-5-trifluoromethoxybenzyl)amino-2-phenyl-piperidine;
(2S,3S)-3-(2-ethoxy-5-trifluoromethoxybenzyl)amino-2-phenyl-piperidine;
(2S,3S)-3-(2-methoxy-5-trifluoromethoxybenzyl)-amino-2-phenylpiperidine;
(2S,3S)-3(-5-tert-butyl-2-trifluoromethoxybenzyl)amino-2-phenylpiperidine;
2-(diphenylmethyl)-N-(2-methoxy-5-trifluoromethoxy-phenyl)methyl-1-azabicyclo[2.2.2]octan-3-amine;
(2S,3S)-3-[5-chloro-2-(2,2,2-trifluoroethoxy)-benzyl]amino-2-phenylpiperidine;
(2S,3S)-3-(5-tert-butyl-2-trifluoromethoxybenzyl)amino-2-phenylpiperidine;
(2S,3S )-3-(2-isopropoxy-5-trifluoromethoxybenzyl)amino-2-phenylpiperidine;
(2S,3S)-3-(2-difluoromethoxy-5-trifluoromethoxybenzyl)-amino-2-phenylpiperidine;
(2S,3S)-2-phenyl-3-[2-(2,2,2-trifluoroethoxybenzyl)-aminopiperidine;
(2S,3S)-2-phenyl-3-(2-trifluoromethoxybenzyl)]aminopi-peridine;
cis-3-(2-chlorobenzylamino)-2-phenylpiperidine;
cis-3-(2-trifluoromethylbenzylamino)-2-phenyl-piperidine;
cis-3-(2-methoxybenzylamino)-2-(2-fluorophenyl)-piperidine;
cis-3-(2-methoxybenzylamino)-2-(2-chlorophenyl)-piperidine;
cis-3-(2-methoxybenzylamino)-2-(2-methylphenyl)-piperidine;
cis-3-(2-methoxybenzylamino)-2-(3-methoxyphenyl)-piperidine;
cis-3-(2-methoxybenzylamino)-2-(3-fluorophenyl)-piperidine;
cis-3-(2-methoxybenzylamino)-2-(3-chlorophenyl)-piperidine;
cis-3-(2-methoxybenzylamino)-2-phenylpiperidine;
cis-3-(2-methoxybenzylamino)-2-(3-methylphenyl)-piperidine;
cis-3-(2-methoxybenzylamino)-2-(4-fluorophenyl)-piperidine;
cis-3-(2-methoxybenzylamino)-2-(3-thienyl)-piperidine;
cis-3-(2-methoxybenzylamino)-2-phenylazacyclo-heptane;
3-(2-methoxybenzylamino)-4-methyl-2-phenylpiperidine;
3-(2-methoxybenzylamino)-5-methyl-2-phenylpiperidine;
3-(2-methoxybenzylamino)-6-methyl-2-phenylpiperidine;
(2S,3S)-3-(2-methoxybenzylamino)-2-phenylpiperidine;
(2S,3S)-1-(5-carboethoxypent-1-yl)-3-(2-methoxybenzyl-amino)-2-phenylpiperidine;
(2S,3S)-1-(6-hydroxy-hex-I-yl)-3-(2-methoxybenzyl-amino)-2-phenylpiperidine;
(2S,3S)-1-(4-hydroxy-4-phenylbut-1-yl)-3-(2-methoxy-benzylamino)-2-phenylpiperidine;
(2S,3S)-1-(4-oxo-4-phenylbut-1-yl)-3-(2-methoxybenzyl-amino)-2-phenylpiperidine;
(2S,3S)-1-(5,6-dihydroxyhex-1-yl)-3-(2-methoxybenzyl-amino)-2-phenylpiperidine;
cis-3-(5-fluoro-2-methoxybenzylamino)-2-phenyl-piperidine;
(2S,3S)-1-[4-(4-fluorophenyl)-4-oxobut-1-yl]-3-(2-methoxybenzylamino)-2-phenylpiperidine;
(2S,3S)-1-[4-[4-fluorophenyl)-4-hydroxybut-1-yl]-3-(2-methoxybenzylamino)-2-phenylpiperidine;
cis-3-(2-methoxy-5-methylbenzylamino)-2-phenyl-piperidine;
(2S,3S)-1-(4-benzamidobut-1-yl)-3-(2-methoxybenzyl-amino)-2-phenylpiperidine;
cis-3-(2-methoxynaphth-1-ylmethylamino)-2-phenyl-piperidine;
(2S,3S)-3-(2-methoxybenzylamino)-1-(5-N-methyl-carboxamidopent-1-yl)-2-phenylpiperidine;
(2S,3S)-1-(4-cyanobut-1-yl)-3-(2-methoxybenzylamino)-2-phenylpiperidine;
(2S,3S)-1-[4-(2-naphthamido)but-1-yl]-3-(2-methoxy-benzylamino)-2-phenylpiperidine;
(2S,3S)-1-(5-benzamidopent-1-yl)-3-(2-methoxybenzyl-amino)-2-phenylpiperidine;
(2S,3S)-1-(5-aminopent-1-yl)-3-(2-methoxybenzylamino)-2-phenylpiperidine;
(2S,3S)-3-(5-chloro-2-methoxybenzylamino)-2-phenyl-piperidine;
(2S,3S)-3-(2,5-dimethoxybenzylamino)-2-phenyl-piperidine;
cis-3-(3,5-difluoro-2-methoxybenzylamino)-2-phenyl-piperidine;
cis-3-(4,5-difluoro-2-methoxybenzylamino)-2-phenyl-piperidine;
cis-3-(2,5-dimethoxybenzylamino)-1-[4-(4-fluorophenyl)-4-oxobut-1-yl]-2-phenylpiperidine;
cis-3-(5-chloro-2-methoxybenzylamino)-1-(5,6-dihydroxyhex-1-yl)-2-phenylpiperidine;
cis-1-(5,6-dihydroxyhex-1-yl)-3-(2,5-dimethoxy-benzylamino)-2-phenylpiperidine;
cis-2-phenyl-3-[-2(prop-2-yloxy)benzylamino]piperidine;
cis-3-(2,5-dimethoxybenzyl)amino-2-(3-methoxy-phenyl)piperidine hydro-chloride;
cis-3-(5-chloro-2-methoxybenzyl)amino-2-(3-methoxy-phenyl)piperidine dihydrochloride;
cis-3-(5-chloro-2-methoxybenzyl)amino-2-(3-chloro-phenyl)piperidine dihydrochloride;3-(2-methoxybenzylamino)-2,4-diphenylpiperidine;
cis-3-(2-methoxybenzylamino)-2-phenylpyrrolidine;
(2S,3S)-3-(5-ethyl-2-methoxybenzyl)amino-2-phenyl-piperidine;
(2S,3S)-3-(5-n-butyl-2-methoxybenzyl)amino-2-phenyl-piperidine;
(2S,3S)-3-(2-methoxy-5-n-propylbenzyl)amino-2-phenyl-piperidine;
(2S,3S)-3-(5-isopropyl-2-methoxybenzyl)amino-2-phenyl-piperidine;
(2S,3S)-3-(5-s-butyl-2-methoxybenzyl)amino-2-phenyl-piperidine;
(2S,3S)-3-(5-t-butyl-2-methoxybenzyl)amino-2-phenyl-piperidine;
(2S,3S)-3-(2-methoxy-5-phenylbenzyl)amino-2-phenyl-piperidine;
2,4-dimethylthiazole-5-sulfonic acid [4-methoxy-3-((2S,3S)-2-phenylpiperidin-3-ylaminomethyl)phenyl]-methylamide;
N-(4,5-dimethylthiazol-2-yl)-N-[4-methoxy-3-((2S,3S)-2-phenylpiperidin-3-yl-aminomethyl)phenyl]-methanesulfonamide;
{5-[(4,5-dimethylthiazol-2-yl)methylamino]-2-methoxybenzyl}-((2S,3S)-2-phenylpiperidin-3-yl)amine;
{5-(4,5-dimethylthiazol-2-ylamino)-2-methoxybenzyl}-((2S,3S)-2-phenylpiperidin-3-ylamine;
4,5-dimethylthiazole-2-sulfonic acid methyl-[3-((2S,3S)-2-phenylpiperidin-3-ylamino-methyl)-4-trifluoromethoxyphenyl]-amide;
2,4-dimethylthiazole-5-sulfonic acid [4-isopropoxy-3-((2S,3S)-2-phenylpiperidin-3-ylaminomethyl)phenyl]-methylamide;
2,4-dimethylthiazole-5-sulfonic acid [4-isopropoxy-3-((2S,3S)-2-phenylpiperidin-3-yl-aminomethyl)phenyl]-isopropylamide;
2,4-dimethylthiazole-5-sulfonic acid [4-methoxy-3-((2S,3S)-2-phenylpiperidin-3-ylamino-methyl)phenyl]-isopropylamide;
2,4-dimethylthiazole-5-sulfonic acid [4-methoxy-3-((2S,3S)-2-phenylpiperidin-3-ylaminomethyl)phenyl]-isobutylamide;
2,4-dimethylthiazole-5-sulfonic acid [4-isopropoxy-3-((2S,3S)-2-phenylpiperidin-3-ylaminomethyl)phenyl]-isobutylamide;
(2S,3S)-N-(5-isopropyl-2-methoxyphenyl)methyl-2-diphenylmethyl-1-azabicyclo [2.2.2]-octan-3-amine;
(2S,3S)-N-(5-tert-butyl-2-methoxyphenyl)methyl-2-diphenyl methyl-1-azabicyclo[2.2.2]octan-3-amine;
(2S,3S)-N-(5-methyl-2-methoxyphenyl)methyl-2-diphenylmethyl-1-azabicyclo[2.2.2]octan-3-amine;
(2S,3S)-N-(5-ethyl-2-methoxyphenyl)methyl-2-diphenyl methyl-1-azabicyclo[2.2.2]octan-3-amine;
(2S,3S)-N-(5-isopropyl-2-methoxyphenyl)methyl-2-diphenylmethyl-1-azabicyclo[2.2.2]octan-3-amine;
(2S,3S)-N-(5-sec-butyl-2-methoxyphenyl)methyl-2-diphenyl methyl-1-azabicyclo[2.2.2]octan-3-amine;
(2S,3S)-N-(5-n-propyl-2-methoxyphenyl)methyl-2-diphenylmethyl-1-azabicyclo[2.2.2]octan-3-amine;
(3R,4S,5S,6S)-N,N-diethyl-5-(5-isopropyl-2-methoxy-benzylamino)-6-diphenylmethyl-1-azabicyclo[2.2.2]octane-3-carboxamide;
(3R,4S,5S,6S)-N,N-diethyl-5-(2,5-dimethoxybenzylamino)-6-diphenylmethyl-1-azabicyclo[2.2.2]octane-3-carboxamide;
(3R,4S,5S,6S)-5-(5-isopropyl-2-methoxybenzylamino)-6-diphenylmethyl-1-azabicyclo[2.2.2]octane-3-carboxylic acid;
(3R,4S,5S,6S)-5-(2-methoxy-2-methylthiobenzylamino)-6-diphenylmethyl-1-azabicyclo[2.2.2]octane-3-carboxylic acid;
(3R,4S,5S,6S)-5-(2,5-dimethoxybenzylamino)-6-diphenyl-methyl-1-azabicyclo-[2.2.2]octane-3-carboxylic acid;
(3R,4S,5S,6S)-5-(2-methoxy-5-methylbenzylamino)-6-diphenylmethyl-1-azabicyclo[2.2.2]octane-3-carboxylic acid;
(3R,4S,5S,6S)-5-(5-ethyl-2-methoxybenzylamino)-6-diphenylmethyl-1-azabicyclo[2.2.2]octane-3-carboxylic acid;
(3R,4S,5S,6S)-5-(2-methoxyl-5-n-propylbenzylamino)-6-diphenylmethyl-1-azabicyclo[2.2.2]octane-3-carboxylic acid;
(3R,4S,5S,6S)-5-(5-sec-butyl-2-methoxybenzylamino)-6-diphenylmethyl-1-azabicyclo[2.2.2]octane-3-carboxylic acid;
(3R,4S,5S,6S)-5-(5-N-methyl-methanesulfonylamino-2-methoxy-benzylamino)-6-diphenylmethyl-1-azabicyclo[2.2.2]octane-3-carboxylic acid;
(3R,4S,5S,6S)-5-(2-methoxy-5-methylsulfinylbenzyl-amino)-6-diphenylmethyl-1-azabicyclo[2.2.2]octane-3-carboxylic acid;
(3R,4S,5S,6S)-5-(2-methoxy-5-trifluoromethoxybenzyl-amino)-6-diphenylmethyl-1-azabicyclo[2.2.2]octane-3-carboxylic acid;
(3R,4S,5S,6S)-5-(2-methoxy-5-methylsulfonylbenzyl-amino)-6-diphenylmethyl-1-azabicyclo[2.2.2]octane-3-carboxylic acid;
(3R,4S,5S,6S)-5-(5-dimethylamino-2-methoxybenzylamino)-6-diphenylmethyl-1-azabicyclo[2.2.2]octane-3-carboxylic acid;
(3R,4S,5S,6S)-5-(5-isopropyl-2-methoxybenzylamino)-6-diphenylmethyl-1-azabicyclo[2.2.2]octane-2-carboxylic acid;
(3R,4S,5S,6S)-5-(2-methoxy-5-methylthiobenzylamino)-6-diphenylmethyl-1-azabicyclo[2.2.2]octane-2-carboxylic acid;
(3R,4S,5S,6S)-5-(2,5-dimethoxy benzylamino)-6-diphenylmethyl-1-azabicyclo[2.2.2]octane-2-carboxylic acid;
(3R,4S ,5S ,6S)-5-(2-methoxy-5-methylbenzylamino)-6-diphenylmethyl-1-azabicyclo[2.2.2]octane-2-carboxylic acid;
(3R,4S,5S,6S)-5-(5-ethyl-2-methoxybenzylamino)-6-diphenylmethyl-1-azabicyclo[2.2.2]octane-2-carboxylic acid;
(3R,4S,5S,6S)-5-(2-methoxyl-5-n-propylbenzylamino)-6-diphenylmethyl-1-azabicyclo[2.2.2]octane-2-carboxylic acid;
(3R,4S,5S,6S)-5-(5-sec-butyl-2-methoxybenzylamino)-6-diphenylmethyl-1-azabicyclo[2.2.2]octane-2-carboxylic acid;
(3R,4S,5S,6S)-5-(5-N-methyl-methanesulfonylamino-2-methoxybenzyl-amino)-6-diphenylmethyl-1-azabicyclo[2.2.2]octane-2-carboxylic acid;
(3R,4S,5S,6S)-5-(2-methoxy-5-methylsulfinylbenzyl-amino)-6-diphenylmethyl-1-azabicyclo[2.2.2]octane-2-carboxylic acid;
(3R,4S,5S,6S)-5-(2-methoxy-5-trifluoromethoxybenzyl-amino)-6-diphenylmethyl-1-azabicyclo[2.2.2]octane-2-carboxylic acid;
(3R,4S,5S,6S)-5-(2-methoxy-5-methylsulfonylbenzyl-amino)-6-diphenylmethyl-1-azabicyclo[2.2.2]octane-2-carboxylic acid; and
(3R,4S,5S,6S)-5-(5-dimethylamino-2-methoxybenzylamino)-6-diphenylmethyl-1-azabicyclo[2.2.2]octane-2-carboxylic acid; or pharmaceutically acceptable salts thereof.

32. The method of claim 20, wherein the NK-1 receptor antagonist is a compound of Formula 1, Formula 2, or a pharmaceutically acceptable salt thereof:

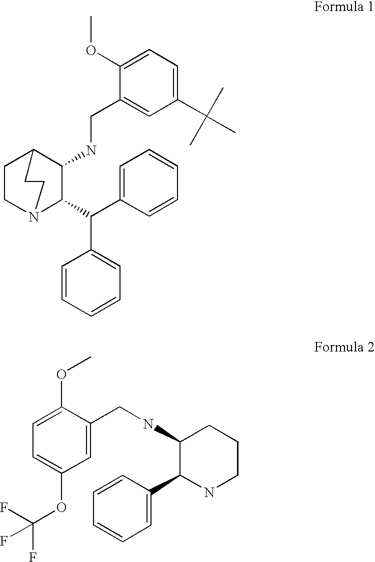

33. The method of claim 20, wherein the NK-1 receptor antagonist is administered once or twice daily for two to four months.
